(19) United States
(12) Patent Application Publication
(10) Pub. No.: US 2010/0209527 A1
(43) Pub. Date: Aug. 19, 2010

(54) COMPOSITION FOR PREVENTION OR TREATMENT OF DISEASE ASSOCIATED WITH PRODUCTION OF AUTOANTIBODY

(76) Inventors: Yoshiya Sato, Okinawa (JP); Kaiisar Mannoor, Okinawa (JP); Isao Shimabukuro, Okinawa (JP); Kikuji Yamaguchi, Shizuoka (JP)

Correspondence Address: BIRCH STEWART KOLASCH & BIRCH, PO BOX 747, FALLS CHURCH, VA, 22040-0747, US

(21) Appl. No.: 12/670,244

(22) PCT Filed: Jul. 23, 2008
(86) PCT No.: PCT/JP2008/063145

§ 371 Date: Jan. 22, 2010

(30) Foreign Application Priority Data
Jul. 24, 2007 (JP) 2007-192148

(51) Int. Cl. A61K 35/64; A61P 37/06

(52) U.S. Cl. 424/537

(57) ABSTRACT

The invention provides a composition for prevention or treatment of a disease associated with the production of an autoantibody.

Specifically, it provides a composition for prevention or treatment of a disease associated with the production of an autoantibody, which comprises royal jelly as an active ingredient.

### TECHNICAL FIELD

The present invention relates to a composition for prevention or treatment of a disease associated with the production of an autoantibody.

### BACKGROUND ART

Autoimmune diseases encompass a variety of conditions wherein the immune response that normally protects an individual is directed to attack the cells or tissue of the individual, and they include many intractable conditions such as rheumatic arthritis, myasthenia gravis and systemic lupus erythematosus (SLE). Many aspects of the mechanisms of autoimmune disease onset remain unelucidated. Treatment therefore involves a dilemma, since immune activity must be suppressed while maintaining resistance, and for this reason fundamental therapeutic methods do not exist at the current time.

It has therefore been desirable to develop a composition for prevention or treatment of diseases associated with the production of autoantibodies.

Royal jelly is a milky yellow-white jelly-like liquid secreted from the cephalic pharyngeal glands and mandibular glands of worker honeybees after consumption of pollen and its metabolism by the heart tube organ, primarily between 3-10 days after eclosion. In honeybee society, royal jelly is provided as a special food for the queen bee. Queen bees fed royal jelly grow to twice the size of the worker bees, and their lifespan is also increased to maintain a long survival period of 3-5 years compared to an average of 35-40 days for worker bees. The queen bee lays as many as 2000-3000 eggs per day during this period, with the honeybees maintaining a highly structured social order.

Royal jelly has been shown to exhibit various physiologically active functions in humans as well, and it has long been considered an important health-maintenance food. It has also been experimentally demonstrated that royal jelly exhibits a notable improving effect on immunomodulation (immune depression) induced by high stress. However, the effect of royal jelly on diseases associated with autoantibody production such as autoimmune diseases has been hitherto unknown.

Royal jelly has been shown to exhibit various physiologically active functions in humans as well, and it has long been considered an important health-maintenance food. It has also been experimental demonstrated that royal jelly exhibits a notable improving effect on immunomodulation (immune depression) induced by high stress. On the other hand, it has been reported that royal jelly contains components that inhibit production of antibodies and cytokines (IL-2, IL-4) and suppress immunological function including antiallergy function (Patent document 1). However, the effect of royal jelly on diseases associated with autoantibody production such as autoimmune diseases has been hitherto unknown.

Royal jelly has been shown to have complex effects that improve the balance between biological functions, including activation of immunological function. Moreover, since royal jelly has already been widely used for years as a health maintenance food or special health food, as is well known, and its safety is adequately assured, it has high practical potential for onset prevention and treatment of autoimmune disease.

[Patent document 1] International Patent Publication No. WO2004/019971

### DISCLOSURE OF THE INVENTION

### Problems to be Solved by the Invention

It is an object of the present invention to provide a composition for prevention or treatment of a disease associated with the production of an autoantibody.

### Means for Solving the Problems

As a result of much diligent research, the present inventors have found, surprisingly, that royal jelly is useful for prevention or treatment of diseases associated with autoantibodies, and the invention has been completed upon this finding.

The present invention therefore provides the following.

1. A composition for prevention or treatment of a disease associated with a production of an autoantibody, which comprises royal jelly as an active ingredient.

2. A composition according to 1 above, wherein the disease associated with production of an autoantibody is selected from the group consisting of Hashimoto's disease, Grave's disease, ulcerative colitis, autoimmune atrophic gastritis, spontaneous Addison's disease, male infertility, autoimmune aspermic testitis, antiglomerular basement membrane, anti-tubular epithelial disease, circulating immune complex-type glomerular nephritis, dermatomyositis, myasthenia gravis, pemphigus vulgaris, bullous pemphigoid, sympathetic ophthalmitis, experimental allergic encephalitis, multiple sclerosis, autoimmune hemolytic anemia, spontaneous thrombocytopenic purpura, spontaneous cardiomyopathy, rheumatic endocarditis, Behcet disease, Sjogren's syndrome, insulin-dependent autoimmune diabetes, non-insulin-dependent diabetes, systemic erythematosus and rheumatoid arthritis.

3. A composition according to claim 2, wherein the disease associated with production of an autoantibody is systemic erythematosus.

### BRIEF DESCRIPTION OF THE DRAWINGS

FIG. 1 shows the effects of royal jelly administration to spontaneous SLE mice. A notable life-lengthening effect was observed in the royal jelly-administered group compared to the control group.

FIG. 2 shows protein concentration in the urine of spontaneous SLE mice treated with royal jelly. Administration of royal jelly produced marked inhibition of proteinuria excretion compared to the control group.

FIG. 3 shows a comparison between antinuclear autoantibody and anti-erythrocyte autoantibody in the royal jelly-administered group and control group. Administration of royal jelly was confirmed to significantly suppress production of autoantibody.

FIG. 4 shows the pathology-improving effect of royal jelly after SLE onset.

### BEST MODE FOR CARRYING OUT THE INVENTION

Autoimmune diseases are disorders in which the cells or tissue of an individual become targets, and their etiology is complex. No effective method of treatment exists other than precise control of the individual's immunological function, and such conditions are therefore intractable. Auto(-responsive) antibodies, or antinuclear antibodies, are usually produced in autoimmune diseases.

Autoimmune diseases are by no means rare, and it has been estimated that approximately 5% of the population are disposed to autoimmune disease, including cases that have not yet reached clinical onset. Most of these are intractable and many are recognized as special diseases in Japan. The onset mechanism is generally believed to involve breakdown of homeostasis of the immune system, but many questions remained unanswered.

Autoimmune diseases are a group of conditions with various modes of onset, wherein the immune response that normally functions to protect the individual is directed to attack the individual's own cells or tissue. The phenomenon in which the immune system is responsible for a pathological state in an organism is generally referred to as “allergy”, and allergies are largely classified into type I allergies caused by hypersensitive reaction to foreign bodies, and autoimmune allergies (type II-IV allergies) wherein the immune system attacks the individual's own cells or tissue. However, the former category (type I allergies, including pollen hypersensitivity, bronchial asthma and atopic dermatitis) and the latter category have fundamentally different onset backgrounds and cannot easily be grouped under the same disease concept. For most cases, therefore, it is more proper for the term “allergy” to refer to the former (type I allergies) and the latter to be classified as autoimmune diseases.

Diseases caused by autoimmunity include a wide range as listed in the following table, and the types are as different as the types of cells and tissues to which the immune response is directed. Among the well-known typical diseases there may be mentioned rheumatic arthritis, myasthenia gravis, systemic lupus erythematosus (SLE), ulcerative colitis (Crohn disease), Behcet disease and non-insulin-dependent diabetes.

TABLE 1
Major autoimmune diseases and their primary target tissues/organs

| Target tissue/organ | Autoimmune disease name |
|---|---|
| Thyroid, parathyroid | Hashimoto's disease, Grave's disease |
| | (Basedow's disease) |
| Stomach, intestines | Ulcerative colitis, autoimmune atrophic |
| | gastritis |
| Adrenal gland | Idiopathic Addison's disease |
| Reproductive system | Male infertility, autoimmune aspermic testitis |
| Kidneys | Antiglomerular basement membrane disease |
| | (Goodpasture's syndrome), anti-tubular |
| | epithelial disease, circulating immune |
| | complex-type glomerular nephritis |
| | (SLE lupus nephritis) |
| Skeletal muscle | Dermatomyositis, myasthenia gravis |
| Skin | Pemphigus vulgaris, bullous pemphigoid |
| Eye ball | Sympathetic ophthalmia |
| Brain/spinal cord/nerves | Experimental allergic encephalitis, multiple |
| | sclerosis |
| Blood components | Autoimmune hemolytic anemia, spontaneous |
| | thrombocytopenic purpura |
| Heart, vascular system | Spontaneous cardiomyopathy, rheumatic |
| | endocarditis |
| Other | Behcet disease, Sjogren's syndrome, insulin- |
| | dependent autoimmune diabetes, non-insulin- |
| | dependent diabetes |

However, autoimmune diseases are by no means rare, and it has been estimated that approximately 5% of the population are disposed to autoimmune disease, including cases that have not yet reached clinical onset. The mechanisms of autoimmune diseases are generally believed to involve breakdown of homeostasis in the immune system, but many aspects of the mechanisms remain unclear. Their treatment also presents a dilemma, since immune activity must be suppressed while maintaining resistance, and for this reason no fundamental therapeutic method has yet been discovered. Most of these are intractable and many are recognized as special diseases in Japan.

Systemic Lupus Erythematosus (SLE):

Systemic lupus erythematosus is one of the representative autoimmune diseases. This is a systemic autoimmune disease preferentially occurring in early adolescent females. In affected patients, erythema is manifest bilaterally on the facial cheeks, and because it appears as butterfly wings, its characteristic symptom is referred to as “butterfly rash”. The pathology of the disease is generally multifaceted, however, and one of the most serious aspects is lupus nephritis. Another feature is production of antinuclear (DNA) autoantibodies and anti-erythrocyte autoantibodies. The mechanism of onset is considered to be mainly type III allergy (antigen/antibody complex formation), although it is also widely believed to be a complex autoimmune disease including type IV allergy. In any case, the onset mechanism is complex and the disease is difficult to treat as progression occurs with repeated aggravation and remission.

The disease associated with production of an autoantibody according to the invention is selected from the group consisting of Hashimoto's disease, Grave's disease, ulcerative colitis, autoimmune atrophic gastritis, spontaneous Addison's disease, male infertility, autoimmune aspermic testitis, antiglomerular basement membrane, anti-tubular epithelial disease, circulating immune complex-type glomerular nephritis, dermatomyositis, myasthenia gravis, pemphigus vulgaris, bullous pemphigoid, sympathetic ophthalmitis, experimental allergic encephalitis, multiple sclerosis, autoimmune hemolytic anemia, spontaneous thrombocytopenic purpura, spontaneous cardiomyopathy, rheumatic endocarditis, Behcet disease, Sjogren's syndrome, insulin-dependent autoimmune diabetes, non-insulin-dependent diabetes, systemic erythematosus and rheumatoid arthritis.

Any conventionally known royal jelly may be used for the present invention. The honeybee type secreting the royal jelly used for the invention may be Apis mellifera, Apis cerana, Apis dorsata, Apis florea or the like.

Locations for production of the royal jelly of the invention include Japan, South America, North America, Australia, China and Europe. These royal jelly products may be unprocessed or treated by appropriate purification steps, and used in any form, purity or preparation method so long as they exhibit an effect of treatment or prevention of a disease associated with production of auto antibody when administered to a human or other mammal.

Moreover, since royal jelly has already been widely used for years as a health maintenance food or special health food, as is well known, its safety is adequately assured.

The composition of the invention may also contain, in addition to royal jelly as the active ingredient, components that are approved for peroral or percutaneous administration or external application onto the skin of humans or other mammals. As examples of such components there may be mentioned water, alcohol, starch, protein, amino acids, fiber, carbohydrates, lipids, fatty acids, vitamins, minerals, flavors, coloring agents, sweeteners, seasonings, spices, antiseptic agents, emulsifiers, surfactants, excipients, extenders, thickeners and preservatives. These components may be used alone or in combinations of two or more.

The composition of the invention may be administered by any pathway known in the prior art, such as peroral or parenteral, for example. The effective amount of consumption or administration for the composition of the invention may be appropriately determined according to the type, age and gender of the target human or other mammal, and for example, it may be ingested or administered perorally, usually at 0.01-100 mg per dosage and preferably 0.1 mg-50 mg per dosage, based on the weight of the active ingredient, per 1 kg of body weight, in one or more doses per day, every day or over an interval of one or more days, depending on the effect.

For production of the composition of the invention, the royal jelly may be combined in an appropriate proportion with one or more components that may be used in the field of foods or beverages, cosmetics, drugs, quasi drugs, feeds, animal provisions, pet foods or the like, in consideration of the type of target animal or the method of ingestion or administration, and appropriate steps such as dilution, concentration, drying, filtration and centrifugal separation may be carried out, with molding into the desired shape, for preparation of a composition comprising an antiallergic drug. There are no particular restrictions to the order of combination of such components and the timing of the steps, so long as the effect of the invention is not impeded.

The composition of the invention may be used in the form of a food or beverage such as a lactic acid beverage or lactic acid bacteria beverage, or in the form of a cosmetic such as a lotion. It may also be used in the form of a drug such as a tablet.

### Examples

The present invention will now be explained in greater detail by concrete experimental examples.

First, aged mice with physiological increased antinuclear autoantibody levels were perorally administered royal jelly to demonstrate significant reduction in antinuclear antibody level. Next, autoimmune disease model mice (NZB×NZWF1:B/W F₁) known to have spontaneous autoimmune disease similar to SLE were administered royal jelly, and a significant life-lengthening effect was found compared to a non-royal jelly-administered control group. Findings indicating onset of autoimmune disease, including urine protein, anti-erythrocyte autoantibody and antinuclear autoantibody levels, were found to be notably suppressed.

### SLE Research Model

As mentioned above, the onset mechanisms of most autoimmune diseases are unclear, and are contrary to the basic principle that the immune system normally does not attack the self. Thus, it is basically difficult to artificially and experimentally induce autoimmune disease, and research is conducted exclusively on experimental mice known as autoimmune disease mice models.

For SLE, New Zealand black mice (NZB) are prepared as SLE disease model mutant mice, and currently the most commonly used are F1 mice crossed with normal New Zealand white mice (NZW), as mice with onset of pathology more closely resembling human SLE. Normally, 50% of the mice exhibit onset of the autoimmune disease and die within 8-9 months of age.

TABLE 2
SLE mouse model condition

| Condition | NZB | NZB * NZW F₁ |
|---|---|---|
| Lupus nephritis | ++ | +++ |
| Vasculitis | ± | + |
| Arthritis | − | − |
| Sialadenitis | + | ++ |
| Hyperimmunoglobulinemia | IgM | IgG |
| Anti-erythrocyte antibody | +++ | + |
| Anti-DNA antibody | IgM | IgG |
| Anti-Sm antibody | − | + |
| Rheumatoid factor | − | − |

The features of NZB×NZW F₁ mice are shown in the table above, and they exhibit pathology which is similar in many respects to human SLE. In addition, the mice have the genetic trait of spontaneous SLE, and most exhibit spontaneous SLE and die within 8-9 months after birth. This mouse model was used for the following experiment.

### Experiment on Preventive Effect of Royal Jelly for SLE Onset

Royal jelly was orally administered to an SLE spontaneous mice model (NZB×NZW F₁; female mice), and the subsequent effect on SLE onset was examined.

Crude royal jelly (provided by Japan Royal Jelly Co., Ltd.) was orally administered to 8-week-old female mice at 0.03 ml (corresponding to 2.0 mg protein) twice a week (Tuesday and Friday), and the subsequent course of SLE onset was observed. A PBS-administered group was provided as a control.

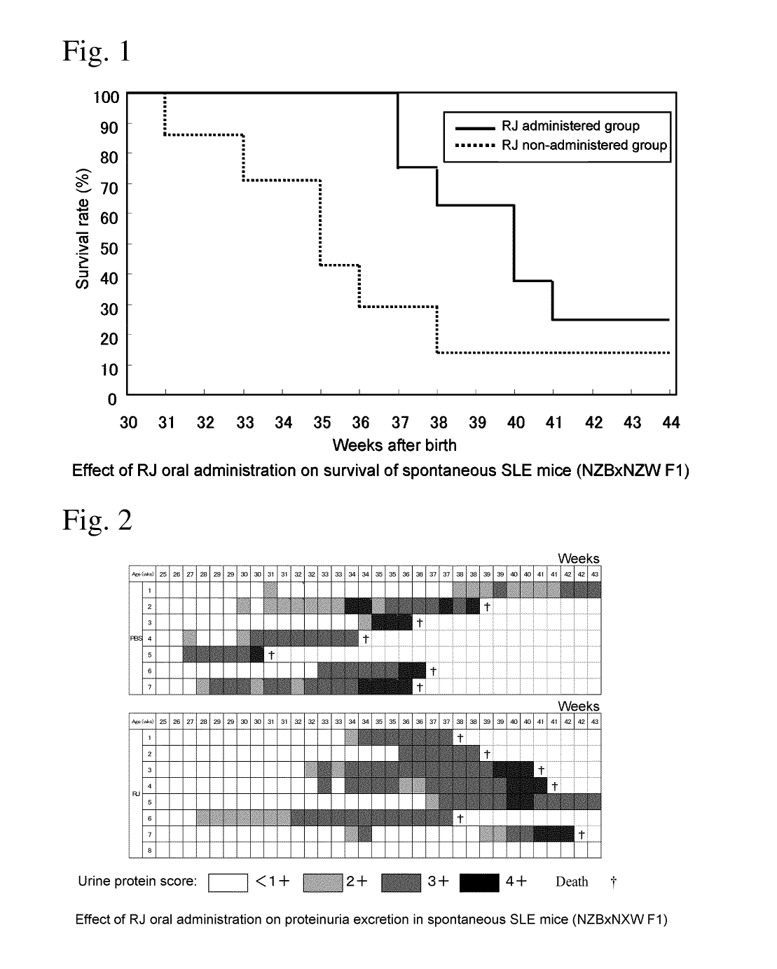

A death curve was drawn for the mice in the royal jelly-administered group and control group (FIG. 1). In the control group, the first death was observed at the 31st week after birth, and all of the individuals except one (6/7) died within 38 weeks after birth. In contrast, this first death was observed at the 37th week after birth in the royal jelly-administered group, with 6 of the 8 mice subsequently dying up to the 41st week. Since a clear difference in death period was seen between the two groups, it was confirmed that royal jelly administration clearly produces a life-lengthening effect for spontaneous SLE mice.

The urine protein was also periodically monitored for mice in the two groups (FIG. 2).

In the control group (PBS-administered group), the mice died prematurely with proteinuria excretion, but in the royal jelly-administered group the proteinuria excretion was notably suppressed, showing a clear link between the life-lengthening effect and the degree of proteinuria.

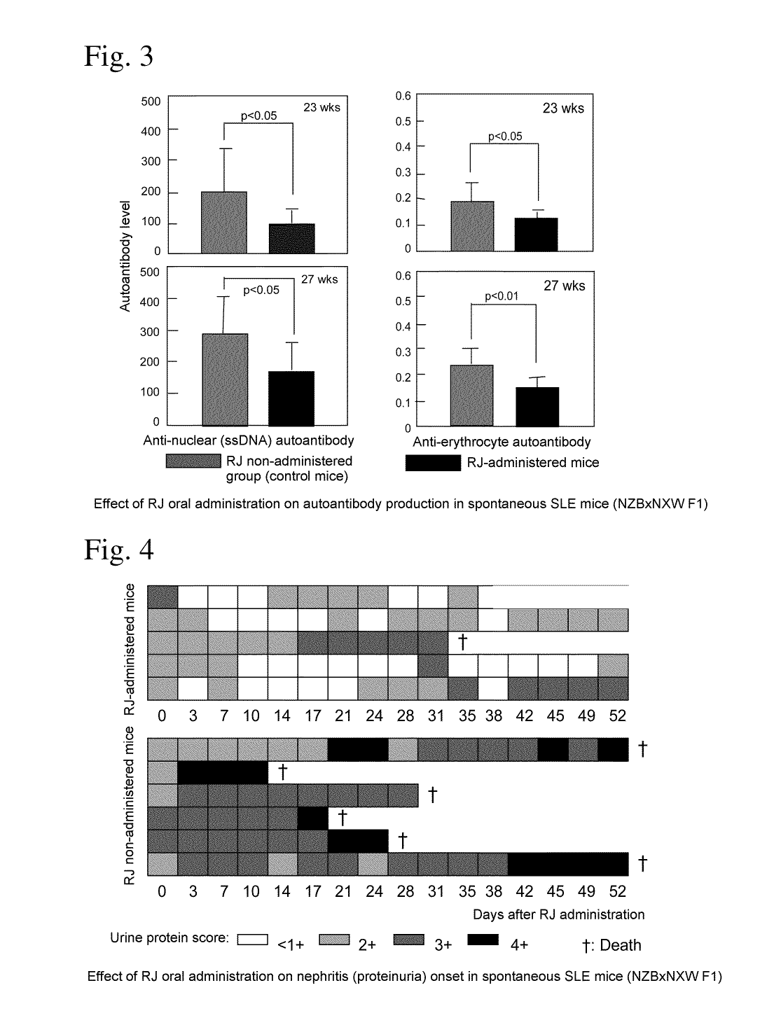

The antinuclear (ssDNA) autoantibody and anti-erythrocyte autoantibody production levels in serum sampled from the mice were also measured (FIG. 3).

In the royal jelly-administered group, notable autoantibody production inhibition was observed before the first death. Since no significant difference in autoantibody levels was found between the two groups during the subsequent period in which deaths of the mice began to occur in both groups, it may be concluded that inhibition of autoantibody production had a direct effect against death of the mice.

High levels of antinuclear (ssDNA) autoantibody and anti-erythrocyte autoantibody are seen for the control group (grey), while notable reduction in both autoantibodies compared to the control group is seen for the royal jelly-administered group.

Pathology-Improving Effect of Royal Jelly after SLE Onset.

The experimental results described above only show the onset-preventing effect of administration of royal jelly to mice models before onset of SLE, and therefore the curative effect of royal jelly after SLE onset was examined next.

In this experiment, urine protein was monitored for mice in two untreated groups, royal jelly was orally administered at the point where urine protein reached 2+ or greater (nephritis onset), and improvement in urine protein excretion was examined thereafter (FIG. 4).

As a result, a notable improving effect on proteinuria excretion was found after administered of royal jelly (0.03 ml, daily oral administration) after appearance of proteinuria.

The following conclusions were drawn:

1) Oral administration of royal jelly (0.03 ml, twice a week) to a spontaneous SLE mice model (NZB×NZW F1) produced a significant life-lengthening effect for the mice.

2) Proteinuria excretion was notably suppressed in the royal jelly-administered mice, thus demonstrating that royal jelly suppresses onset of nephritis.

3) In addition, anti-DNA autoantibody and anti-erythrocyte autoantibody production was inhibited in these mice, demonstrating that royal jelly inhibits expression of the autoimmune response.

4) In addition, administration of royal jelly (0.03 ml, daily) after nephritis onset (after appearance of urine protein) temporarily improved proteinuria.

5) These results clearly showed that royal jelly has an effect of preventing onset of the autoimmune disease SLE, and improving symptoms after onset.

### INDUSTRIAL APPLICABILITY

The invention is useful for prevention or treatment of diseases associated with production of autoantibody, such as systemic erythematosus.

1-3. (canceled)

4. A method for treating or preventing an autoimmune disease comprising administering an effective amount of a composition comprising a royal jelly to a subject.

5. The method of claim 4, wherein the autoimmune disease is Hashimoto's disease, Grave's disease, ulcerative colitis, autoimmune atrophic gastritis, spontaneous Addison's disease, male infertility, autoimmune aspermic testitis, antiglomerular basement membrane, anti-tubular epithelial disease, circulating immune complex-type glomerular nephritis, dermatomyositis, myasthenia gravis, pemphigus vulgaris, bullous pemphigoid, sympathetic ophthalmitis, experimental allergic encephalitis, multiple sclerosis, autoimmune hemolytic anemia, spontaneous thrombocytopenic purpura, spontaneous cardiomyopathy, rheumatic endocarditis, Behcet disease, Sjogren's syndrome, insulin-dependent autoimmune diabetes, non-insulin-dependent diabetes, systemic lupus erythematosus or rheumatoid arthritis.

6. The method of claim 4, wherein the autoimmune disease is systemic lupus erythematosus.

7. The method of claim 4, wherein the royal jelly is secreted by Apis mellifera, Apis cerana, Apis dorsata, or Apis florae.

8. The method of claim 4, wherein the composition is administered perorally, topically, or parenterally.

9. The method of claim 4, wherein the composition further comprises at least one of water, an alcohol, a starch, a protein, an amino acid, fiber, a carbohydrate, a lipid, a fatty acid, a vitamin, a mineral, a flavor, a coloring agent, a sweetener, a seasoning, a spice, an antiseptic agent, an emulsifier, a surfactant, an excipient, an extender, a thickener, and a preservative.

10. The method of claim 4, wherein the composition is administered at least once daily such that 0.01 to 100 mg of royal jelly per kg of the subject's body weight is delivered with each administration.

11. The method of claim 4, wherein the composition is administered at least once daily such that 0.01 to 50 mg of royal jelly per kg of the subject's body weight is delivered with each administration.

12. The method of claim 4, wherein the composition is in the form of a food, a beverage, a cosmetic, a drug, a quasi drug, a feed, a pet food, or an animal provision.

13. The method of claim 4, wherein the composition is in the form of a lactic acid beverage or a lactic acid bacteria beverage.

14. The method of claim 4, wherein the composition is in the form of a lotion.

15. The method of claim 4, wherein the subject is a human or a non-human animal.

16. The method of claim 4, wherein the subject has been diagnosed with systemic lupus erythematosus.
